(19) United States
(12) Patent Application Publication
(10) Pub. No.: US 2006/0062747 A1
(43) Pub. Date: Mar. 23, 2006

(54) Use of sweetener acids for the microbiological stabilization of foodstuffs, cosmetic products, consumer goods and pharmaceutical productions

(75) Inventors: Susanne Rathjen, Darmstadt (DE); Gerhard Merkt, Frankfurt (DE)

Correspondence Address: PROPAT, L.L.C., 425-C SOUTH SHARON AMITY ROAD, CHARLOTTE, NC, 28211-2841, US

(73) Assignee: Nutrinova Nutrition Specialties & Food Ingredients GmbH, Frankfurt am Main (DE)

(21) Appl. No.: 10/534,205

(22) PCT Filed: Nov. 11, 2003
(86) PCT No.: PCT/EP03/12567

§ 371 Date: May 6, 2005

(30) Foreign Application Priority Data
Nov. 19, 2002 (DE) 102 53 773.9
Jul. 3, 2003 (DE) 103 30 026.0

(51) Int. Cl. A61K 8/60; A61K 8/49; A61K 31/704; A61K 31/425

(52) U.S. Cl. 424/70.1; 514/26; 514/373; 514/27

(57) ABSTRACT

The invention relates to the use of sweetener acids for the microbiological stabilization of foodstuffs, cosmetic products, consumer goods and pharmaceutical products, in particular in foodstuffs, beverages, pharmaceutical and cosmetic products, the intensity of the acidic taste of said acids being lower than that of the quantity of a conventional food acid that is required to achieve the pH value reduction. The invention also relates to an agent containing at least one sweetener acid and at least one highly concentrated sweetener.

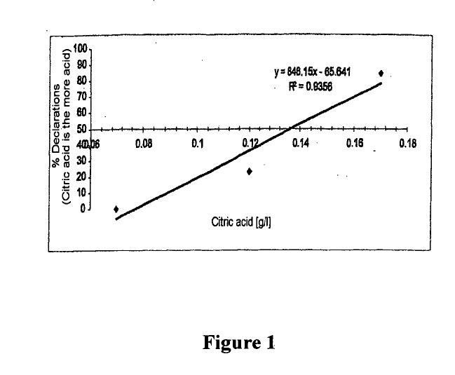

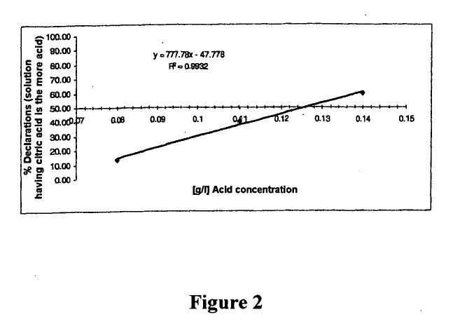

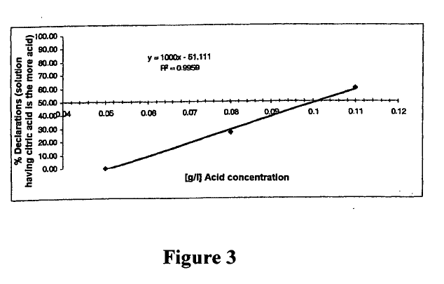

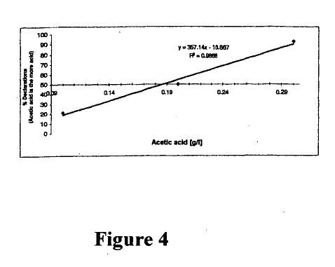

The present invention relates to the use of sweetener acids for the microbiological stabilization of foods, cosmetics, consumer goods and pharmaceuticals, in particular in foods, drinks, pharmaceuticals and cosmetics, the intensity of the acid taste being less than in the case of the amount of a conventional food acid which is necessary to achieve the same pH reduction. Furthermore, the invention relates to a composition comprising a sweetener acid and at least one high-intensity sweetener.

High-intensity sweeteners are compounds of synthetic or natural origin which have no physiological calorific value, or a negligible physiological calorific value in relation to the sweetening power, and have a sweetening power many times higher than sucrose. High-intensity sweeteners are used in foods and drinks individually or in combination with the purpose of causing a sweet taste.

Acidulents are constituents in foods and drinks which contribute to a number of tastes, microbiological and/or technological functions and properties. Acidulents are divided into organic and inorganic acidulents. The acidulents customarily used in the food and drinks sector include the organic acids adipic acid, malic acid, succinic acid, acetic acid, fumaric acid, glucono-delta-lactone and gluconic acid, lactic acid, tartaric acid, citric acid, and also the inorganic acid phosphoric acid. These acidulents give foods and drinks a more or less characteristic acid taste note. The acid basic taste is triggered by the H⁺ or H₃O⁺ ions produced by dissociation of the acid in an aqueous medium. The phenomenon of the intensity of the acid taste is, however, not explained scientifically. Since the intensity of the acid taste of various acids is not correlated with the acid strength (acid constant) (see table 1), other factors such as concentration, pH and the specific anion of the acidulent appear to play a critical role. In particular, an effect on the intensity of the acid taste is ascribed to the ability of the anions to penetrate or bind to the receptor membrane. At identical concentrations, the intensity of the acid taste decreases in the following sequence: fumaric acid>tartaric acid>malic acid>acetic acid>citric acid>lactic acid>gluconic acid.

TABLE 1
Taste profile and acid strength of acidulent

| Acid | Taste characteristics of food acids | pKₐ1 | pKₐ2 | pKₐ3 |
|---|---|---|---|---|
| Acetic | Strong volatile acid, vinegar-like | 4.75 | — | — |
| acid | odor and astringent taste | | | |
| Adipic | Acid taste, but without sharpness, | 4.43 | 5.41 | — |
| acid | persistent chalky note | | | |
| Citric | Sharp, clean acid taste with only | 3.09 | 4.77 | 6.39 |
| acid | brief residence time on the gums | | | |
| Fumaric | Strong, metallic acid taste with long | 3.03 | — | — |
| acid | residence time on the gums | | | |
| Gluconic | Weak acid taste | 3.7 | — | — |
| acid | | | | |
| Lactic | Mild acid taste, but with long | 3.86 | — | — |
| acid | residence time on the gums | | | |
| Malic acid | Strong but soft acid taste with | 3.4 | 5.05 | — |
| | relatively long residence time on the | | | |
| | gums | | | |
| Phosphoric | Raw, biting flat acid taste, | 2.12 | 7.21 | ˜12.4 |
| acid | persistent | | | |
| Tartaric | Sharp and bitter acid taste of short | 2.98 | 4.34 | — |
| acid | duration | | | |

In addition, the various acidulents have a different taste profile which critically affect their use in foods and drinks (see table 1). Citric acid, the organic acidulent most frequently used in the drinks sector, has, for example, a rapidly starting acid taste which does not persist long. Malic acid, in contrast, is distinguished by later starting and longer persistence of the acid taste.

In addition to the use of acidulents in foods and drinks for sensory reasons, acidulents are used for pH reduction and the resultant inhibition of microorganisms. The pH optimum of most food-spoilage and food-poisoning bacteria is pH 5-8. While most food-poisoning bacteria have their pH minimum at pH 4.5 and thus can be inhibited in many foods by addition of acidulents alone, many food-spoilage microorganisms such as lactic acid bacteria and acetic acid bacteria and also yeasts and molds are significantly more acid tolerant. Frequently, for the preservation of foods and drinks, acidulents are used in combination with other methods of preservation, such as chemical preservation, and also biological and physical methods, to build up cumulative inhibition effects. For instance, the effect of chemical preservation of foods and drinks using sorbic acid or benzoic acid is amplified by pH reduction using acidulents.

The pH of foods and drinks given via acidulents has a critical effect on technological properties of foods and drinks, beyond the taste and its microbiological significance. Via the pH, acidulents can stabilize the color of the product, change turbidity, melting and flow behavior, and also affect the foam formation, gel formation and emulsion behavior of foods. Furthermore, these acids can also act as blowing agents or emulsifiers in foods and drinks. As what are termed synergists, acidulents reinforce the action of antioxidants by complexing catalytic heavy metal ions.

Acidulents, which are customarily used for acidification and pH reduction of the taste of drinks and foods, increase the intensity of the acid taste and change the aroma profile of the drinks and foods to be acidified. The change in the aroma profile can, in addition to general superimposition by the basic acid taste, also be caused by the specific non-acid taste properties of the acidulent, for example as in the case of acetic acid (see table 1).

In foods and drinks in which such changes in taste caused by addition of an acidulent are not desired, or adversely affect consumer acceptance, when the abovementioned acidulents which are conventional on the market are used, pH reduction sufficient for microbiological or technical reasons cannot be performed. These acidulents which are conventional on the market and which have a comparatively softer or milder taste, for example lactic acid or gluconic acid, are also weaker acidulents (see table 1), which either results in a lower pH reduction, or in a higher usage concentration, in order to achieve the desired pH.

It was therefore an object of the present invention to provide a food additive which effects a marked pH reduction in the food, cosmetic, consumer good or pharmaceutical, without impairing this product too greatly in sensory terms. The inventive composition, therefore, is to reduce the pH, in particular in foods and drinks, the intensity of the acid taste being less than in the case of the amount of a conventional food acid which is necessary to achieve the same pH reduction.

This object is achieved by using sweeteners for pH reduction in foods, pharmaceuticals, consumer goods and cosmetics, in particular in foods, pharmaceuticals and cosmetics, particularly preferably in drinks, table sweeteners and dairy products, the intensity of the acid taste being less than in the case of the amount of a conventional food acid which is necessary to achieve the same pH reduction.

Sweetener acids are the acids of known salts of high-intensity sweeteners such as acesulfame-K (=potassium salt of acesulfamic acid), sodium cyclamate or sodium saccharin. Sweetener acids which can be used are, for example, saccharin acid, cyclamic acid, glycyrrhicic acid and acesulfamic acid and also mixtures of two or more of these acids. Inventively preferred sweetener acids are acesulfamic acid, cyclamic acid and saccharin acid, and also mixtures of two or all three sweetener acids. On account of their low pKₐ of 1.5 to 2.5, the sweetener acids have never previously been considered as sweeteners. If sweetener acids are used in foods and drinks, surprisingly, in the sensory testing, it has been found that sweetener acids, despite their property as strong acids, and as a result marked pH reduction potential, have only a low acid intensity in taste. The acid profile is balanced. The time-intensity profile of the acid taste of, for example, acesulfamic acid, is comparable to that of malic acid (see table 1).

At the same time, the sweetener acids have, on a molar basis, a sweetening power equivalent to the corresponding sweetener salt. In addition to the described acid taste and sweet taste, no significant off-taste and aftertaste occur.

Sweetener acids are obtained from sweetener salt production by omitting the step of neutralizing the sweetener acid with a base. Sweetener acids, however, can also be produced from the commercially available sweetener salts by acidification, for example by sulfuric acid. The sweetener acid is then extracted from the acidic solution by an organic solvent such as ethyl acetate and is then isolated, for example, by evaporating off the solvent.

Acesulfamic acid is obtained, for example, by what is termed the SO₃ method, as described in EP-A-0 155 634, and by which acesulfame-K is also produced. The acid is obtained after ring closure using SO₃, before neutralization using potassium hydroxide. However, acesulfamic acid can also be produced from the commercially available acesulfame-K by acidification, for example using sulfuric acid. Acesulfamic acid is then extracted from the acidic solution by an organic solvent such as ethyl acetate and is then isolated, for example by evaporating off the solvent.

According to the invention, the sweetener acids are used in foods, expediently in amounts of 20 to 5000 ppm, preferably in amounts of 40 to 2000 ppm, in particular in amounts of 50 to 1000 ppm (in each case based on the mass of the food or drink used).

In cosmetics, consumer goods and pharmaceuticals, the inventive sweetener acids are expediently used in amounts of 20 to 12 000 ppm, preferably in amounts of 40 to 8000 ppm, in particular in amounts of 50 to 5000 ppm (in each case based on the mass of the cosmetic, consumer good or pharmaceutical used).

The use of sweetener acids as sweeteners and acidulents in drinks and foods causes a pH reduction and makes possible the use of accompanying microbiological and/or technological advantages with a lesser effect on the intensity of the acid taste and the aroma profile than with the use of commercially conventional acidulents. For an identical acid taste, thus a significantly lower pH can be achieved using sweetener acids than using commercially conventional acidulents. Preferably, for an identical acid taste, the pH can be reduced by 0.2 to 0.6 units. This effect is relevant to drinks and also to all foods in which, for abovementioned microbiological or technological reasons, a pH reduction is to be achieved, for example soft drinks, preferably “aromatized waters”, or what are termed “near water” or “flavored water” products, fruit juice drinks, jams and jellies, fruit preserves and vegetable preserves, desserts, delicatessen products, sauces, table sweeteners. With equally good effect, sweetener acids may be used in pharmaceuticals and cosmetics.

The invention further relates to a composition comprising at least one sweetener acid and at least one high-intensity sweetener.

High-intensity sweeteners according to the invention are taken to mean sweeteners such as aspartame, alitame, neotame, acesulfame-K, saccharin, cyclamat, sucralose, thaumatin, neohesperidin dihydrochalcone (NHDC), neotame and stevioside. Preferred high-intensity sweeteners are aspartame, alitame, neotame, acesulfame-K, saccharin, cyclamate and sucralose.

In the inventive composition, the weight ratio between sweetener acid and high-intensity sweetener is expediently between 100:1 and 1:20, preferably 50:1 and 1:10, particularly preferably 20:1 and 1:5, and in particular preferably 1:1 to 1:2.

When neotame is used as high-intensity sweetener in the inventive mixture, the weight ratio between sweetener acid and high-intensity sweetener can also be 1000:1 to 1:1, preferably 500:1 to 1:1, and in particular preferably 250:1 to 1:1.

The invention will be described in more detail hereinafter with reference to examples.

### EXAMPLES

Use of Sweetener Acids in Drinks for pH Reduction:

A sweetener acid very frequently used in drinks is citric acid. The effect of sweetener acids on the pH and the acid impression compared with citric acid is described hereinafter. Since the perception of the intensity of sweetness and acidity affect each other, all citric acid solutions having one of the sweetener acid concentrations used were admixed with an equimolar and equisweet concentration of the respective corresponding salt of the sweetener acid. The concentration of the citric acid solution was set depending on the objective, for example pH equivalence or equivalence of the acid intensity.

### Example 1

### Acesulfamic Acid as Sweetener Acid

a) Determination of the Relative Sweetener Intensity at the Same pH

Method:

Solution A: Acesulfamic acid (Nutrinova, Frankfurt, Germany) (203 mg/l) and
Solution B: Acesulfame-K (Sunett®) (250 mg/l), set to the same pH as solution A using citric acid

Triangle test (DIN ISO 4120 (January 1995)) with the question: Which sample is the more acid? (forced choice, n=12)

Result:

At an identical pH setting, solution A is rated less acid than solution B by 10 of 12 testers. Solution B achieves a very highly significantly stronger acid taste compared with solution A (significance level=0.1%). This is clearly a specific effect of acesulfamic acid, since in the test systems equal amounts of the acesulfame anion were present. In summary, the results are shown in table 2.

TABLE 2
Triangle test for determining relative acid intensity at an identical pH

| | Solution A | Solution B |
|---|---|---|
| Acesulfamic acid [mg/l] | 203 | — |
| Citric acid monohydrate [mg/l] | — | 590 |
| Acesulfame-K [mg/l] | — | 250 |
| pH | 3.07 | 3.03 |
| Number of testers who perceived | 2 | 10 |
| the sample as more acid | | |

b) Identical Acid Taste Test of Acesulfamic Acid Against Citric Acid
Method:
Test solution: Acesulfamic acid (203 mg/l)
Standard A: Acesulfame-K (Sunett®) (250 mg/l)+citric acid (0.07 g/l)
Standard B: Acesulfame-K (Sunett®) (250 mg/l)+citric acid (0.12 g/l)
Standard C: Acesulfame-K (Sunett®) (250 mg/l)+citric acid (0.17 g/l)

Pairwise comparison test with the question: Which sample is the more acid: (forced choice, n=13)

Result:

[image]

An aqueous solution having 203 mg/l of acesulfamic acid has an identical acid taste of a solution consisting of 137 mg/l of citric acid+250 mg/l of acesulfame-K. The acesulfamic acid solution has a significantly lower pH of 3.07 than the citric acid solution (pH 3.51). In summary, the results are shown in table 3.

TABLE 3
Identical acid taste solutions of acesulfamic
acid and citric acid

| | Acesulfamic | Citric acid + |
|---|---|---|
| | acid | acesulfame-K |
| Acesulfamic acid [mg/l] | 203 | — |
| Citric acid.H₂O [mg/l] | — | 137 |
| Acesulfame-K [mg/l] | — | 250 |
| pH | 3.07 | 3.51 |

### Example 2

### Cyclamic Acid as Sweetener Acid

a) Determination of the Relative Sweetness Intensity at an Identical pH

Method:

Solution A: Cyclamic acid, N-cyclohexylsulfaminic acid, (No. 29550, Fluka, Germany) (180 mg/l) and
Solution B: Sodium cyclamate (No. 817044, Merck-Schuchardt, Germany) (202 mg/l), set to the same pH as solution A by citric acid

Triangle test (DIN ISO 4120 (January 1995)) with the question: Which sample is the more acid? (forced choice, n=12)

Result:

At an identical pH setting, solution A is rated as less acid than solution B by 12 of 12 testers. Solution B achieves a very highly significantly stronger acid taste compared with solution A (significance level=0.1%). This is clearly a specific effect of cyclamic acid, since in the test systems identical amounts of the cyclamate anion were present. In summary, the results are presented in table 4.

TABLE 4
Triangle test for determining the relative
acid intensity at an identical pH

| | Solution A | Solution B |
|---|---|---|
| Cyclamic acid [mg/l] | 180 | — |
| Citric acid monohydrate [mg/l] | — | 590 |
| Sodium cyclamate [mg/l] | — | 202 |
| pH | 3.09 | 3.04 |
| Number of testers who perceived | 0 | 12 |
| the sample as more acid | | |

b) Identical Acid Taste Test of Cyclamic Acid Against Citric Acid
Method:
Test solution: Cyclamic acid (180 mg/l)
Standard A: Sodium cyclamate (202 mg/l)+citric acid (0.08 g/l)
Standard B: Sodium cyclamate (202 mg/l)+citric acid (0.11 g/l)
Standard C: Sodium cyclamate (202 mg/l)+citric acid (0.14 g/l)

Pairwise comparison test with the question: Which sample is the more acid? (forced choice, n=15)

Result:

[image]

An aqueous solution having 180 mg/l of cyclamic acid has identical acid taste to a solution consisting of 126 mg/l of citric acid+202 mg/l of sodium cyclamate. The cyclamic acid solution has a significantly lower pH of 3.08 than the citric acid solution (pH 3.45). In summary, the results are shown in table 5.

TABLE 5
Solutions of identical acid taste of cyclamic
acid and citric acid

| | Cyclamic | Citric acid + sodium |
|---|---|---|
| | acid | cyclamate |
| Cyclamic acid [mg/l] | 180 | — |
| Citric acid.H₂O [mg/l] | — | 126 |
| Sodium cyclamate [mg/l] | — | 202 |
| pH | 3.08 | 3.45 |

### Example 3

### Saccharin Acid as Sweetener Acid

a) Determination of the Relative Sweetness Intensity at an Identical pH

Method:

Solution A: Saccharin acid, o-benzosulfimide (No. 12475, Fluka, Germany) (200 mg/l) and
Solution B: Saccharin sodium (No. 817042 S20913 711, Merck-Schuchardt, Germany) (225 mg/l), set to the identical pH as solution A by citric acid

Triangle test (DIN ISO 4120 (January 1995)) with the question: Which sample is the more acid? (forced choice, n=12)

Result:

At an identical pH setting, solution A is rated less acid than solution B by 12 of 12 testers. Solution B achieves a very highly significantly stronger acid taste in the comparison with solution A (significance level=0.1%). This is clearly a specific effect of saccharin acid, since identical amounts of the saccharin anion were present in the test systems. In summary, the results are shown in table 6.

TABLE 6
Triangle test for determining the relative acid
intensity at an identical pH

| | Solution A | Solution B |
|---|---|---|
| Saccharin acid [mg/l] | 200 | — |
| Citric acid monohydrate [mg/l] | — | 590 |
| Saccharin sodium [mg/l] | — | 225 |
| pH | 3.04 | 3.03 |
| Number of testers who perceived | 0 | 12 |
| the sample as more acid | | |

b) Identical Acid Taste Determination of Saccharin Acid in Comparison with Citric Acid
Method:
Test solution: Saccharin acid (200 mg/l)
Standard A: Saccharin sodium (225 mg/l)+citric acid (0.05 g/l)
Standard B: Saccharin sodium (225 mg/l)+citric acid (0.08 g/l)
Standard C: Saccharin sodium (225 mg/l)+citric acid (0.11 g/l)

Pairwise comparison test with the question: Which sample is the more acid? (forced choice, n=15)

Result:

[image]

An aqueous solution having 200 mg/l of saccharin acid is of identical acid taste to a solution consisting of 100 mg/l of citric acid+225 mg/l of saccharin sodium. The saccharin acid solution has a significantly lower pH of 3.05 than the citric acid solution (ph 3.53). In summary, the results are shown in table 7.

TABLE 7
Identical acid taste solutions of saccharin acid and citric acid

| | Saccharin | Citric acid + saccharin |
|---|---|---|
| | acid | sodium |
| Saccharin acid [mg/l] | 200 | — |
| Citric acid.H₂O [mg/l] | — | 100 |
| Saccharin sodium [mg/l] | — | 225 |
| pH | 3.05 | 3.53 |

### Example 4

### Foods

Acetic acid is frequently used as fruit acid in the acidification of foods, for example delicatessen products, and other acidic preserved products. In particular in the case of pickled vegetables, the typical acetic acid peak is rounded off by using sugar or high-intensity sweeteners, to give the product a higher acceptance with consumers. In addition, a (partial) replacement of the acetic acid by the combined sweetener and acidulent acesulfamic acid is of economic relevance, since the desired technological (pH) and sensory properties of the product can be achieved by a lower total usage rate of acidulent and sugar or sweetener.

The effect of the sweetener/acidulent acesulfamic acid on the pH and acid impression compared with sweetener solutions based on the salt of acesulfamic acid (acesulfame-K) and acetic acid may be described as follows:

a) Determination of Relative Sweetness Intensity at an Identical pH

Method:

Solution A: Acesulfamic acid (203 mg/l) and
Solution B: Acefulfame-K (250 mg/l) set to the identical pH as solution A by acetic acid.

Triangle test with the question: Which sample is the more acid (forced choice, n=12)

Result:

At an identical pH setting, solution A having the sweetener/acidulent acesulfamic acid is rated by 12 of 12 testers as less acid than solution B, consisting of acesulfame-K and acetic acid. Solution B achieves a very highly significantly stronger acid taste compared with solution A (significance level=0.1%). This is clearly a specific effect of the acesulfamic acid, since, in the test systems, equal amounts of the acesulfame anion were present. In summary, the results are shown in table 8.

TABLE 8
Triangle test determining relative acid intensity at an identical pH

| | Solution A | Solution B |
|---|---|---|
| Acesulfamic acid [mg/l] | 203 | — |
| Acetic acid [mg/l] | — | 2930 |
| Acesulfame-K [mg/l] | — | 250 |
| pH | 3.07 | 3.08 |
| Number of testers who perceived | 0 | 12 |
| the sample as more acid | | |

b) Identical Acid Taste Determination of Acesulfamic Acid Against Acetic Acid
Method:
Test solution: Acesulfamic acid (203 mg/l)
Standard A: Acesulfame-K (250 mg/l)+acetic acid (0.1 g/l)
Standard B: Acesulfame-K (250 mg/l)+acetic acid (0.2 g/l)
Standard C: Acesulfame-K (250 mg/l)+acetic acid (0.3 g/l)
Pairwise comparison test with the question: Which sample is the more acidic? (forced choice, n=14)
   Result:

[image]

An aqueous solution having 203 mg/l of acesulfamic acid is of identical acid taste to a solution consisting of 190 mg/l acetic acid+250 mg/l of acesulfame-K. The acesulfamic acid solution has a significantly lower pH of 3.07 than the citric acid solution (pH 3.71). In summary, the results are shown in table 9.

TABLE 9
Identical acid taste solutions of acesulfamic acid and acetic acid

| | Acesulfamic | Acetic acid + |
|---|---|---|
| | acid | acesulfame-K |
| Acesulfamic acid [mg/l] | 203 | — |
| Acetic acid [mg/l] | — | 190 |
| Acesulfame-K [mg/l] | — | 250 |
| pH | 3.07 | 3.71 |

### Example 5

### Use of the Sweetener Acid Acesulfamic Acid in Combination with the Sweetener Aspartame, Compared with the Use of Citric Acid as Acidulent

A mixture of 175 mg/l of acesulfamic acid and 150 mg/l of aspartame was produced in an orange-aroma-containing drink based on water. The pH was 3.4; the acid perception corresponded to a comparable orange-aroma-containing drink, but acidified using 0.1 g/l of citric acid instead of acesulfamic acid, in which case the pH set itself to 3.9. To obtain a drink of identical sweetness, 214 mg/l of Sunett and 150 mg/l of aspartame were used.

### Example 6

### Use of the Sweetener Acids Acesulfamic Acid and Cyclamic Acid in Combination with the Sweetener Neotame in Comparison with the Use of Citric Acid as Acidulent

A mixture of 100 mg/l of cyclamic acid, 150 mg/l of acesulfamic acid and 1 mg/l of neotame was produced in an iced peach tea. The pH was 3.5; the acid perception corresponded to a comparable iced peach tea, but acidified using 0.12 g/l of citric acid instead of cyclamic acid and acesulfamic acid, in which case the pH set itself to 4.2. To obtain a drink of identical sweetness, 185 mg/l of Sunett, 112/l of Na cyclamate and 1 mg/l of neotame were used.

### Example 7

### Use of the Sweetener Acids Acesulfamic Acid and Cyclamic Acid in Combination with the Sweetener Alitame in Comparison with the Use of Citric Acid as Acidulent

A mixture of 100 mg/l of cyclamic acid, 150 mg/l of acesulfamic acid and 5 mg/l of alitame was produced in an iced lemon tea. The pH was 3.5; the acid perception corresponded to a comparable iced lemon tea, but acidified with 0.12 g/l of citric acid instead of cyclamic acid and acesulfamic acid, in which case the pH set itself to 4.2. To obtain a drink of identical sweetness, 185 mg/l of Sunett and 112 mg/l of Na cyclamate and 5 mg/l of alitame were used.

1. A microbial stabilizer comprising a sweetener acid and one or more of either a food, a cosmetic, a consumer good and a pharmaceutical.

2. A microbial stabilizer as claimed in claim 1, wherein said stabilizer comprises food.

3. A microbial stabilizer as claimed in claim 1, wherein said stabilizer comprises a pharmaceutical and/or cosmetic.

4. A microbial stabilizer as claimed in claim 2 wherein the sweetener acid is used in an amount of about 20 to 5000 ppm.

5. A microbial stabilizer as claimed in claim 3, wherein the sweetener acid is used in an amount of 20 to 12 000 ppm.

6. A microbial stabilizer as claimed in claim 1, wherein the sweetener acid is selected from one or more of the following sweetener acids: acesulfamic acid, saccharin acid, cyclamic acid and glycyrrhicic acid.

7. A microbial stabilizer as claimed in claim 1, wherein said stabilizer completely or partially replaces food acids present within one or more of either a food a cosmetic a consumer good and a pharmaceutical.

8. A composition comprising at least one sweetener acid and at least one high-intensity sweetener.

9. The composition as claimed in claim 8, wherein the sweetener is selected from one or more compounds selected from the group consisting of aspartame, alitame, neotame, acesulfame-K, saccharin, cyclamate, sucralose, thaumatin, neohesperidin dihydrochalcone, neotame and stevioside.

10. The composition as claimed in claim 8, wherein the sweetener acid is selected from one or more of the following sweetener acids: acesulfamic acid, saccharin acid, cyclamic acid and glycyrrhicic acid.

11. The composition as claimed in claim 8, wherein the weight ratio between sweetener acid and high-intensity sweetener is between 1000:1 and 1:20.

12. A method for the identical acid taste reduction of pH, which comprises replacing an existing acid wholly or in part by a sweetener acid.

13. The method as claimed in claim 12, wherein the pH is reduced in a food, drink, pharmaceutical or cosmetic.

14. The method as claimed in claim 12, wherein the acid to be replaced is a food acid.

15. A drink comprising a composition as claimed in claim 8.

16. A food comprising a composition as claimed in claim 8.

17. A pharmaceutical comprising a composition as claimed in claim 8.

18. A cosmetic comprising a composition as claimed in claim 8.
